(19) United States
(12) Patent Application Publication
(10) Pub. No.: US 2024/0270694 A1
(43) Pub. Date: Aug. 15, 2024

(54) PROCESS FOR THE PREPARATION OF IVACAFTOR AND NOVEL SOLVATES THEREOF

(71) Applicant: RK Pharma Inc., Pearl River, NY (US)

(72) Inventors: Ravishanker Kovi, North Brunswick, NJ (US); Murali Krishna Madala, Hyderabad (IN); Jayaraman Kannappan, Village Dhobikuva (IN); Hemant M. Mande, Village Dhobikuva (IN); Sureshkumar Kanagaraj, Hyderabad (IN); Moorkonda Raja Sekhar, Hyderabad (IN)

(73) Assignee: RK Pharma Inc., Pearl River, NY (US)

(21) Appl. No.: 18/462,619

(22) Filed: Sep. 7, 2023

(30) Foreign Application Priority Data
Sep. 7, 2022 (IN) 202241051153

(51) Int. Cl. C07D 215/56

(52) U.S. Cl. CPC C07D 215/56

(57) ABSTRACT

The present invention provides industrially applicable, commercially viable, eco-friendly processes for the preparation of Ivacaftor. The present invention also provides novel solvates of Ivacaftor.

### CROSS REFERENCE TO RELATED APPLICATION(S)

This application claims priority to Indian Application No. 202241051153, filed Sep. 7, 2022. The entirety of the disclosure of the above-referenced application is incorporated herein by reference.

### FIELD OF THE INVENTION

The present invention provides industrially applicable, commercially viable, eco-friendly processes for the preparation of Ivacaftor. The present invention also provides novel solvates of Ivacaftor.

### BACKGROUND OF THE INVENTION

Ivacaftor is a drug used to treat cystic fibrosis in people with certain mutations in the cystic fibrosis transmembrane conductance regulator (CFTR) gene, who account for 4-5% cases of cystic fibrosis. It is also included in a combination drug, Lumacaftor/Ivacaftor (trade name Orkambi) which is used for the treatment of cystic fibrosis (CF) in patients aged 1 year and older who have two copies of the F508del mutation (F508del/F508del) in their CFTR gene. More recently, a new combination of Tezacaftor/Ivacaftor (trade name Symdeco) has been approved by USFDA.

Ivacaftor was approved by FDA and is marketed by Vertex Pharmaceuticals under the brand name KALYDECO®. Ivacaftor is chemically known as N-(2,4-di-tert-butyl-5-hydroxyphenyl)-1,4-dihydro-4-oxoquinoline-3-carboxamide, having molecular weight 392.49 and formula C₂₄H₂₈N₂O₃. Ivacaftor has the following structural Formula I:

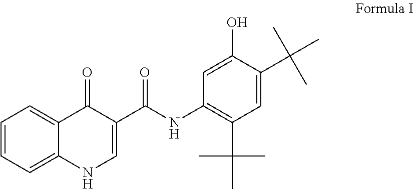 [SMILES: CC(C)(C)c1cc(C(C)(C)C)c(NC(=O)c2c[nH]c3ccccc3c2=O)cc1O]

Ivacaftor was first referred in U.S. Pat. No. 7,495,103. However, Ivacaftor process was not specifically disclosed. The '103 patent disclosed that heating a mixture of aniline of Formula 2 and diethyl 2-(ethoxymethylene)malonate of Formula 3 at 140-150° C. to afford 2-phenylamino methylene-malonic acid diethyl ester of Formula 4, which further treated with polyphosphoric acid and phosphoryl chloride to form ethyl 1,4-dihydro-4-oxoquinoline-3-carboxylate of Formula 5. Further hydrolysed to form 1,4-dihydro-4-oxoquinoline-3-carboxylic acid of Formula 6, then on condensation with 2,4-di-tert-butyl-5-aminophenol of Formula 7 in presence of a coupling reagent such as 1-[bis(dimethylamino)methylene]-1H-1,2,3-triazolo[4,5-b]pyridinium-3-oxidehexafluorophosphate (HATU), N, N-diisopropylethylamine (DIPEA) in dimethylformamide and purifying the obtained compound by preparative HPLC.

This process for the preparation of Ivacaftor disclosed heating at very high temperature (140-150° C.), which is not feasible for commercial scale production. Moreover, the process involves the use of high quantity of polyphosphoric acid and phosphoryl chloride, which generates huge quantity of effluent and large amount of impurities, which are difficult to remove or require successive purifications to get the pure compound. Therefore, yield obtained of Ivacaftor is low. In-addition of this, large quantity of base will be required to neutralize the high quantity of acidic effluent generated.

U.S. Pat. No. 8,476,442B2 discloses a process for the preparation of Ivacaftor by heating a mixture of aniline of Formula 2 and diethyl 2-(ethoxymethylene)malonate of Formula 3 at 100-110° C. to afford 2-phenylamino methylene-malonic acid diethyl ester of Formula 4, which further treated in presence of phenyl ether at 228-232° C. to form ethyl 1,4-dihydro-4-oxoquinoline-3-carboxylate of Formula 5. Subsequently, the compound of Formula 5 is hydrolysed to form 1,4-dihydro-4-oxoquinoline-3-carboxylic acid of Formula 6, which on further reaction with 5-amino-2,4-di-tertbutylphenyl methyl carbonate of Formula 8 in the presence of propane phosphonic anhydride and pyridine using 2-Methyltetrahydrofuran gives hydroxy protected Ivacaftor of Formula 9. Finally, the compound of Formula 9 is deprotected using NaOMe/MeOH in presence of 2-Methyltetrahydrofuran to obtain Ivacaftor.

This process for the preparation of Ivacaftor is cumbersome, which involves many number of steps and costlier reagent like propane phosphonic anhydride, NaOMe and pyridine. The strong unpleasant odour of pyridine is not conducive to industrial application. Further, it causes the health hazards to the plant chemists/workers. The unavoidable inhalation of pyridine causes dizziness, headache, insomnia, ataxia, nervousness, loss of appetite, gastrointestinal dysfunction, and in turn, liver and kidney damage can occur. It can also cause dermatitis. Also, use of ethereal solvent such as 2-Methyltetrahydrofuran is not preferred on commercial scale due to the cost and safety of the same. This process involves heating at higher temperature 100-110° C. and 228-232° C., due to which intermediate compounds 4 and 5 respectively are formed with large amount of impurities.

The drawback of above prior art is increased number of steps/unit operations, in turn increases the cost of chemicals, utilities, occupancy of the plant, requirement of manpower and hence the cost of the production.

IN4022/MUM/2015 discloses a process for the preparation of compound of Formula 6 by coupling diethyl 2-(ethoxymethylene)malonate of Formula 3 with aniline of Formula 2 in the presence of isopropyl alcohol to obtain compound of Formula 4. Further treating with polyphosphoric acid in the presence of sulfolane at 102±3° C. Then hydrolyse with sodium hydroxide to obtain compound of Formula 6. This process involves the use of additional solvent isopropyl alcohol and higher temperature 102±3° C. In addition, the solvent used sulfolane is costlier and more toxic as compare to other solvents. Therefore, process is relatively less economical, hence, not suitable for commercial production.

Therefore, the prior art processes are relatively costly, unsafe and hence, not suitable for commercial production.

Many other patent publications have also been disclosed so far, which describe the process for the preparation of Ivacaftor. Still there is a consequently a need for an alternative method for the preparation of Ivacaftor and new crystalline forms and new processes for preparing crystalline forms. The said methods should be more industrially scalable, economic, should consist of reagents which are cheaper, easy to handle, and allows only desired compounds to be obtained with high purity yields. The present invention relates to various novel solvates of Ivacaftor.

### SUMMARY OF THE INVENTION

In one aspect, the present invention provides an improved process for the preparation of Ivacaftor formula I or a pharmaceutically acceptable salt thereof, comprising the steps of a) coupling of compound of Formula ICA-03, with compound of Formula ICB-02 or a salt thereof to provide compound of Formula I Ivacaftor.

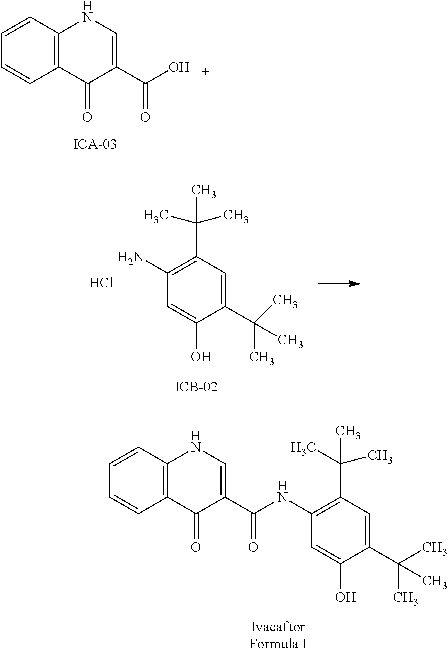 [SMILES: O=C(O)c1c[nH]c2ccccc2c1=O ; Cl ; CC(C)(C)c1cc(C(C)(C)C)c(O)cc1N ; CC(C)(C)c1cc(C(C)(C)C)c(NC(=O)c2c[nH]c3ccccc3c2=O)cc1O]

using suitable base and reagent in the presence of suitable solvent to form Ivacaftor; and optionally, purifying by suitable method.

In another aspect, the present invention provides a purification of Ivacaftor of Formula-I.

In an aspect, the present invention provides crystalline Ivacaftor n-butyl acetate solvate.

In another aspect, the present invention provides crystalline Ivacaftor n-butyl acetate solvate characterized by X-ray diffraction spectrum having peaks at 4.88, 5.15, 7.86, 8.34, 9.64, 11.0, 13.17, 18.7, 19.49, 19.77, 22.69±0.2° 2θ.

In a further aspect, the present invention also provides crystalline Ivacaftor Diethyl ether, Anisole, 1,2-dimethoxyethane, 2-ethoxy ethanol, Trifluoroacetic acid, Chlorobenzene, Diphenyl ether, Bromobenzene, Iodobenzene, Benzonitrile, 1,1,2-trichloroethane, Carbon tetrachloride, 2,2-dimethoxypropane, Methyl isopropyl ketone, 2-Methyl THF solvates.

In another aspect, the present invention provides polymorphic forms RK1, RK2, RK3, RK4, RK5, RK6, RK7, RK8, RK9, RK10, RK11, RK12, RK13, RK14, RK15, RK16, RK17, of Ivacaftor and process for the preparation thereof.

### BRIEF DESCRIPTION OF DRAWINGS

FIG. 1: Represents X-ray powder diffraction pattern of Ivacaftor Form RK1.

FIG. 2: Represents DSC (Differential Scanning calorimetry) of Ivacaftor Form RK1.

FIG. 3: Represents TGA (Thermogravimetric analysis) of Ivacaftor Form RK1.

FIG. 4: Represents NMR (Nuclear Magnetic Resonance) spectrogram of Ivacaftor Form RK1.

### DETAILED DESCRIPTION OF THE INVENTION

The present invention provides an efficient and industrially advantageous process for the preparation of Ivacaftor, and novel solvates thereof.

In accordance with one embodiment, the present invention provides a process for the preparation of Ivacaftor comprising the steps;

a) involves coupling aniline of formula 2 with diethyl 2-(ethoxymethylene)malonate of formula 3 in absence of the solvent to afford diethyl [(phenylamino)methylidene]propanedioate of formula ICA-01. Reaction was carried out at a temperature of 100-110° C. for 4-5 hours.

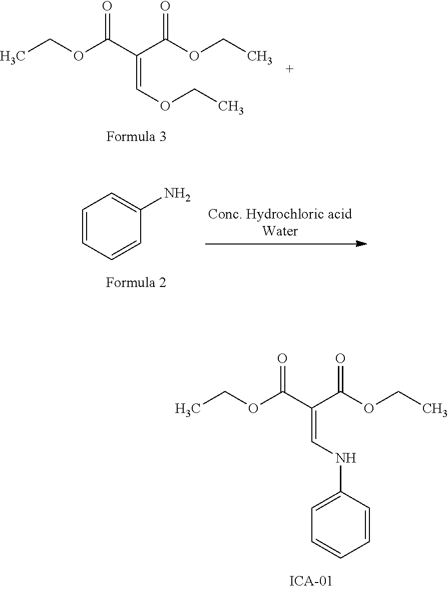 [SMILES: CCOC=C(C(=O)OCC)C(=O)OCC ; Nc1ccccc1 ; CCOC(=O)C(=CNc1ccccc1)C(=O)OCC]

In further purifying the obtained compound of formula ICA-01, comprises
1) adding into a solvent and cooled to 5-10° C.,
2) adjust the pH 1-2 using HCl
3) filtering the material and washing with solvent,
4) drying the material at 25-35° C. for 12-14 hours.

b) cyclizing the compound of formula ICA-01 using a suitable cyclizing agent in the absence of solvent, to provide compound of formula ICA-02. Reaction was carried out at a temperature of 95-100° C. for 4-5 hours.

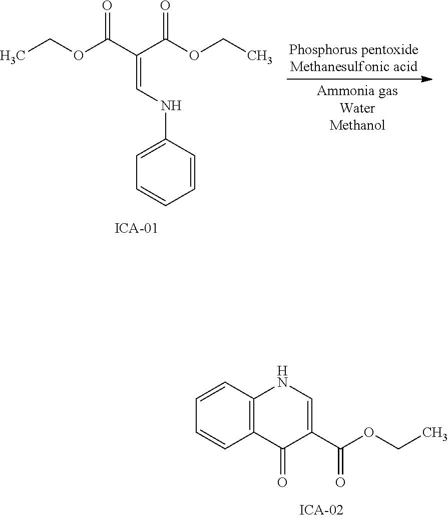 [SMILES: CCOC(=O)C(=CNc1ccccc1)C(=O)OCC ; CCOC(=O)c1c[nH]c2ccccc2c1=O]

Suitable reagent used for cyclization in step b) includes Eaton's reagent (Phosphorus pentoxide in methanesulfonic acid), polyphosphoric acid, and phosphorous oxychloride. Preferably Eaton's reagent (Phosphorus pentoxide in methanesulfonic acid).

According to the embodiment, the Eaton's reagent is used for cyclization reaction and it is best to control the side chain reactions, wherein happen in prior art processes. Aforesaid prior art POCl₃ reagent is used a large quantity for cyclization. Excess of this reagent is required a huge amount of base for quenching and it leads to impurities, which is difficult to remove in further purification steps. Moreover, Eaton's reagent is cheaper, commercially and economically available.

The obtained compound of formula ICA-02, further purified by, comprising
1) adding into a solvent, heating to 50-55° C. and stir for one hour.
2) cooling the mixture to 25-35° C. and stir for 2 hours.
3) filtering the solid material and washing with solvent.
4) drying at 50-55° C. for 12-14 hours.

c) involves hydrolysing compound of formula ICA-02 in presence of base to form compound of formula ICA-03. Hydrolysis carried out at 85-90° C. for 4-5 hours.

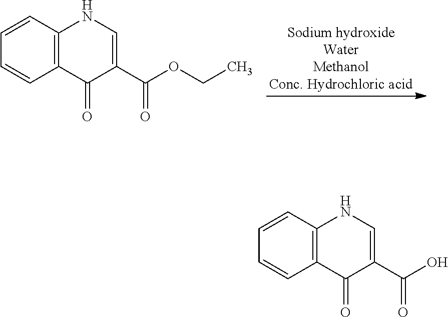 [SMILES: CCOC(=O)c1c[nH]c2ccccc2c1=O ; O=C(O)c1c[nH]c2ccccc2c1=O]

The obtained compound of formula ICA-03, further purified by, comprising
1) adding the Compound ICA-03 into a solvent and heating to 45-50° C.
2) Stirring for 30-60 minutes
3) cooling the resultant mixture to 10-15° C. and stirring for 2 hours
4) filtering the obtained solid and washing with solvent
5) drying the wet material at 45-50° C. for 8-10 hours

d) involves coupling of compound of formula ICA-03 with compound of formula ICB-02 using suitable reagent and base in presence of solvent to form Ivacaftor. Coupling may be carried out at 45-50° C. for 4-5 hours.

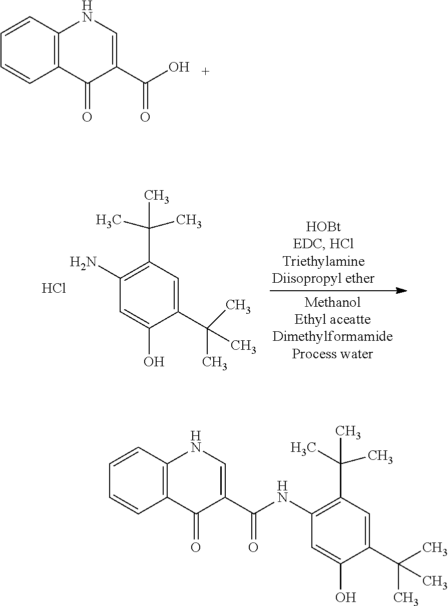 [SMILES: O=C(O)c1c[nH]c2ccccc2c1=O ; Cl ; CC(C)(C)c1cc(C(C)(C)C)c(O)cc1N ; CC(C)(C)c1cc(C(C)(C)C)c(NC(=O)c2c[nH]c3ccccc3c2=O)cc1O]

The suitable reagent used in step-d) is selected from group comprising of 1-[bis(dimethylamino)methylene]-1H-1,2,3-triazolo[4,5-b]pyridinium-3-oxide hexafluorophosphate (HATU), N,N,N′,N′-tetramethyl-O-(1H-benzotriazol-1-yl)uranium hexafluorophosphate (HBTU), Hydroxybenzotriazole (HOBt), N,N′-Dicyclohexylcarbodimide (DCC), 1-(3-dimethylaminopropyl)-3-ethylcarbodiimide hydrochloride (EDC.HCl), Propanephosphonic acid anhydride (PPAA, T3P), N-ethoxycarbonyl-2-ethoxy-1,2-dihydroquinoline (EEDQ), 1,1′-Carbonyldiimidazole (CDI), N-hydroxysuccinimide (HOSu), (benzotriazol-1-5yloxy)tris(dimethylamino)phosphonium hexafluorophosphate (BOP), isobutyl chloroformate (IBCF), O-(1H-6-chlorobenzotriazole-1-yl)-1,1,3,3-tetramethyluronium hexafluorophosphate (HCTU) or the like and mixture thereof. Preferably, reagent used is HOBt.

The suitable solvent used in step d) is selected from group comprising of water, sulfoxides, alcohols, halogenated hydrocarbons, ethers, esters, amides, hydrocarbons such as dimethyl sulfoxide (DMSO), methanol, ethanol, n-propanol, isopropanol, n-butanol, dichloromethane (DCM), chloroform, dichloroethane, chlorobenzene, diethyl ether, methyl tert-butyl ether (MTBE), diisopropyl ether, tetrahydrofuran (THF), 2-methyltetrahydrofuran, dioxane, methyl acetate, ethyl acetate, butyl acetate, isopropyl acetate, N,N-dimethylformamide (DMF), N,N-dimethylacetamide (DMAc), N-methylformamide, N-methylpyrrolidone, pentane, hexane, heptane, octane, cyclohexane, cyclopentane, toluene, xylene or the like and mixture thereof, preferably, solvent used is N,N-dimethylformamide (DMF).

The base used during condensation in step d) may be selected from inorganic or organic base, wherein inorganic base is selected from alkali or alkaline earth metal hydrides, hydroxides, carbonates, bicarbonates, sodium with liquid ammonia, sodamide or like and mixture thereof; organic bases may be selected from diisopropylethylamine, triethylamine, tributylamine, N-methylmorpholine, 1,8-diazabicyclo[5.4.0]undec-7-ene (DBU) and pyridine or the like and mixture thereof, preferably, base used is triethylamine.

In another embodiment, the present invention provides a process for the preparation of 5-amino-2,4-ditert-butyl-phenol hydrochloride of formula ICB-01.

The process comprises,

1) bromination of 2,4-ditert-butyl phenol of formula II in presence of solvent to obtain a compound of formula ICB-01A, followed by protection of hydroxy group with methyl chloroformate formula III to obtain hydroxy protected compound of formula ICB-01B.

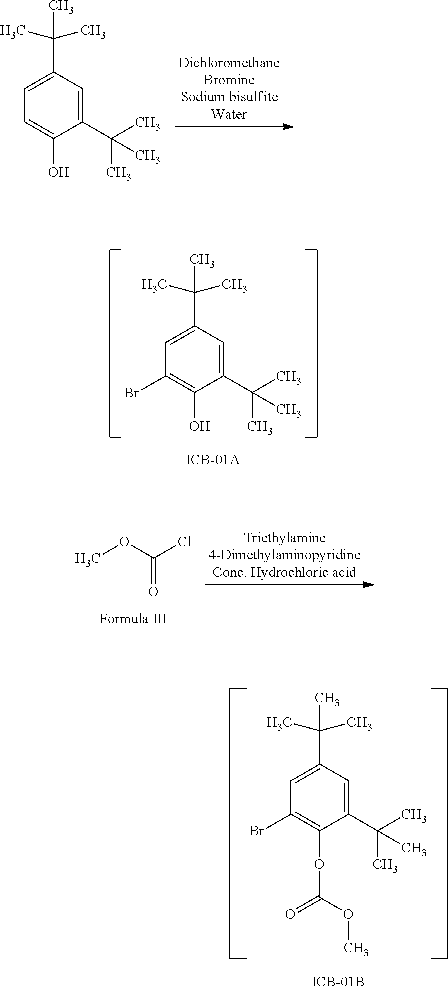

2) Nitration with the mixture of sulfuric acid and nitric acid to obtain 6-bromo-2,4-ditert-butyl-5-nitrophenyl methyl carbonate of formula ICB-01 and then isolating formula ICB-01 by using methanol solvent, wherein the reaction is carried out without isolating formula ICB-01A to ICB-01B from the reaction mixture.

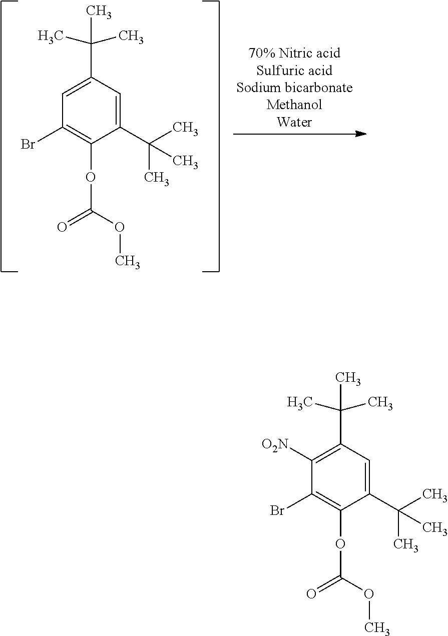

In another embodiment, present invention provides a process for purification of compound of formula ICB-01, which comprises
1) dissolving the Compound ICB-01 in methanol, heating to 55-60° C. and stirring for 2 hours.
2) cooling the resultant mixture to 0-5° C. and stirring for 2-3 hours.
3) filtering the solid and washing with a solvent.
4) drying the wet material at 50-55° C. for 8-10 hours.

3) The process comprises, reduction of compound of formula ICB-01 in presence of reducing agent to form compound of formula ICB-02A without isolating compound of formula ICB-02A in presence of base to form compound of formula ICA-02B is not isolated.

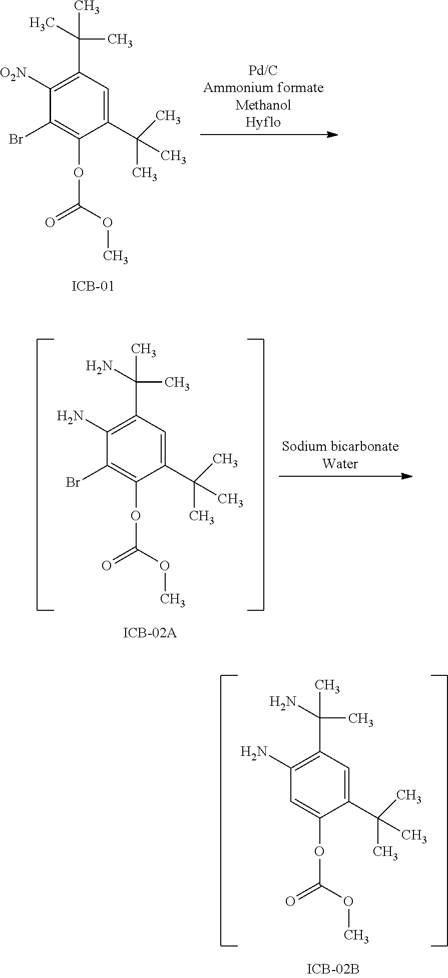

The suitable reducing agent used in reduction of formula ICB-01 and ICB-02A comprises, Palladium on Carbon, Platinum on Carbon, Iron in HCl, Iron/NH₄Cl, SnCl₂, Sodium hydrosulfite, Tin(II) chloride, Zinc/NH₄Cl, Zn/hydrazine hydrate, Raney nickel, sodium bisulfite, NaBH₄ and the like.

The suitable based used in debromination of ICB-2A comprises, Sodium bicarbonate, Sodium acetate (anhydrous), Sodium acetate trihydrate, Sodium carbonate, Potassium bicarbonate, Potassium acetate and the like.

The solvent used in step 1) is selected from the group consisting of alcohols, ethers, esters, water, ketones, halogenated hydrocarbons, nitriles, amides and mixtures thereof; preferably, the solvent used is Dichloromethane. The solvent used in step 3) is alcohols, preferably Methanol.

4) Then the Deprotection of the compound of formula ICB-02B is carried out in presence of suitable acid to obtain compound of formula ICB-02.

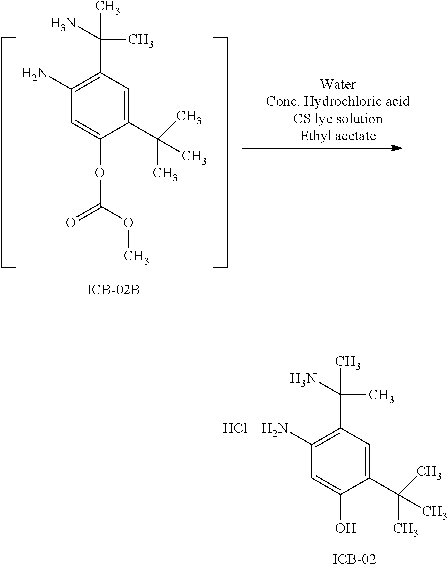

The suitable deprotecting agent used herein for the Deprotection of compound of formula ICB-02B, such as hydrochloric acid, hydrobromic acid, trifluoroacetic acid preferably hydrochloric acid.

The suitable solvent for Deprotection of the compound of formula ICB-02, methanol, ethanol, acetone, methyl isobutyl ketone, water, tetrahydrofuran, methyl tertiary butyl ether. Preferably water.

The Deprotection reaction may be carried out at a temperature of about 5° C. to reflux temperature. Preferably at 5-35° C. and most preferably at 20-35° C.

In a further embodiment of the invention, present invention provides process for the purification of compound of formula ICB-02, which comprises,
1) dissolving the Compound ICB-02 in a solvent and heating to 60-65° C. for 1 hour.
2) Cooling the resultant mixture to 5-15° C. and stirring for 2 hours.
3) filtering the solid and washing with a solvent.
4) drying the wet material at 60-65° C. for 8-10 hours.

Obtained compound of formula ICB-02 with a compound of formula ICA-03 in presence of coupling agent, base to provide Ivacaftor compound of formula I.

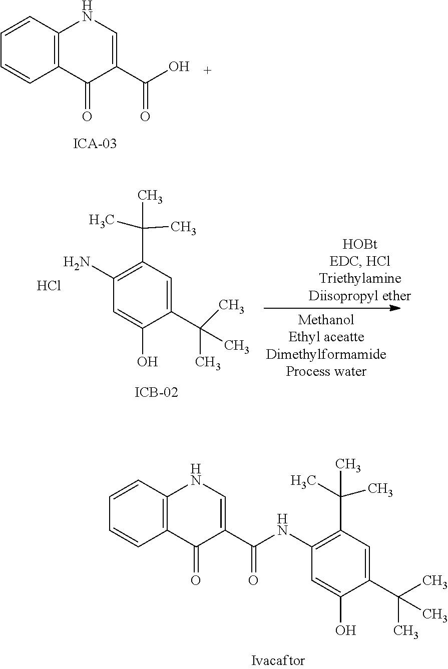 [SMILES: O=C(O)c1c[nH]c2ccccc2c1=O ; Cl ; CC(C)(C)c1cc(C(C)(C)C)c(O)cc1N ; CC(C)(C)c1cc(C(C)(C)C)c(NC(=O)c2c[nH]c3ccccc3c2=O)cc1O]

In an illustrated embodiment, the present invention provides a process for purification of Ivacaftor, which comprises:
1) dissolving Ivacaftor or a solvate thereof in an organic solvent,
2) adding another solvent to the reaction mass of step 1 and heated the reaction mixture and stir for 2 hours.
3) cooling the reaction mass; and
4) isolating the Pure Ivacaftor.

The solvent used in step 1) is selected from the group consisting of alcohols, ethers, esters, ketones, halogenated hydrocarbons, nitriles, amides and the like, and mixtures thereof; preferably the solvent used is alcohol, most preferably methanol.

The solvent used in step 2) is selected from the group consisting of esters, most preferably Ethyl acetate.

In a further embodiment, the present invention provides novel polymorphic forms of Ivacaftor, process for the preparation of such polymorphic forms.

In an embodiment, the present invention provides Ivacaftor solvates such as n-butyl acetate, Diethyl ether, Anisole, 1,2-dimethoxyethane, 2-ethoxy ethanol, Trifluoroacetic acid, Chlorobenzene, Diphenyl ether, Bromobenzene, Iodobenzene, Benzonitrile, 1,1,2-trichloroethane, Carbon tetrachloride, 2,2-dimethoxypropane, Methyl isopropyl ketone and 2-Methyl THF.

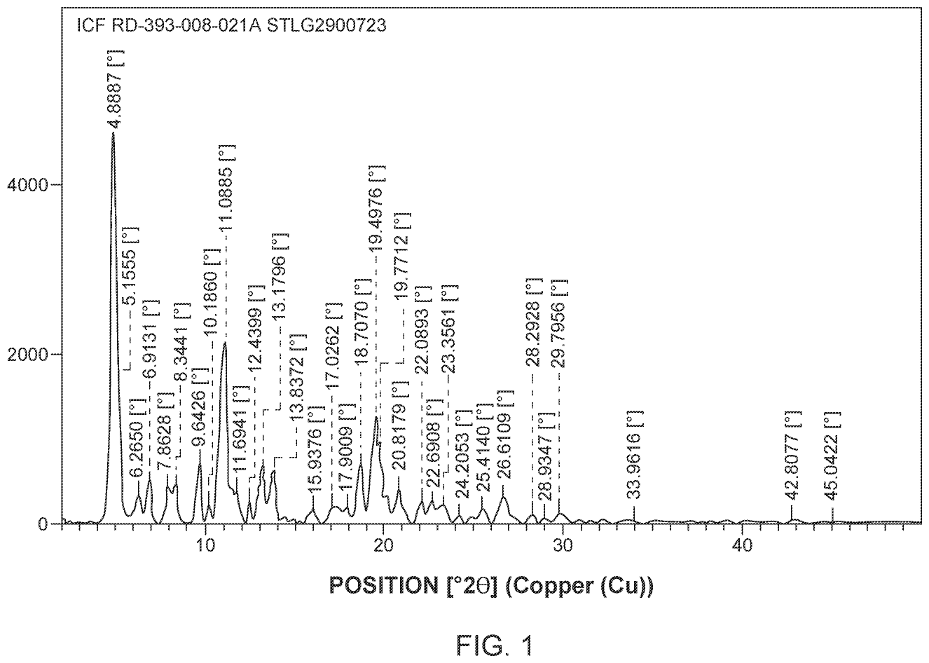

In another embodiment, the present invention provides Ivacaftor polymorphic form RK1 characterized by a powder X-Ray diffraction (PXRD) pattern substantially in accordance with FIG. 1.

In another embodiment, the present invention provides Ivacaftor polymorphic form RK1 characterized by a PXRD pattern having one or more peaks at about 4.88, 5.15, 7.86, 8.34, 9.64, 11.08, 13.17, 13.83, 18.70, 19.49, 19.77, 20.81, 22.69±0.2° 2θ.

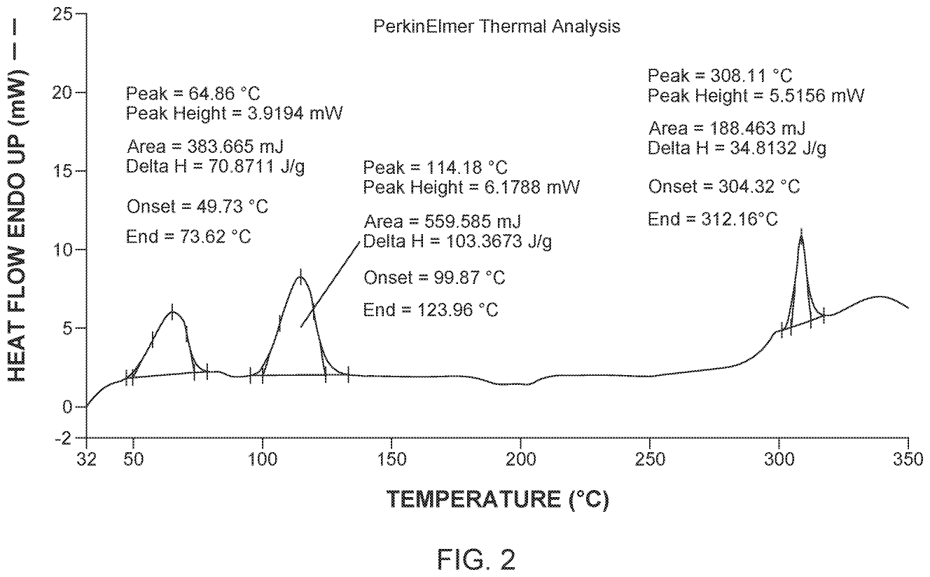

In another embodiment, the present invention provides Ivacaftor polymorphic form RK1 characterized by a differential scanning calorimetry (DSC) substantially in accordance with FIG. 2.

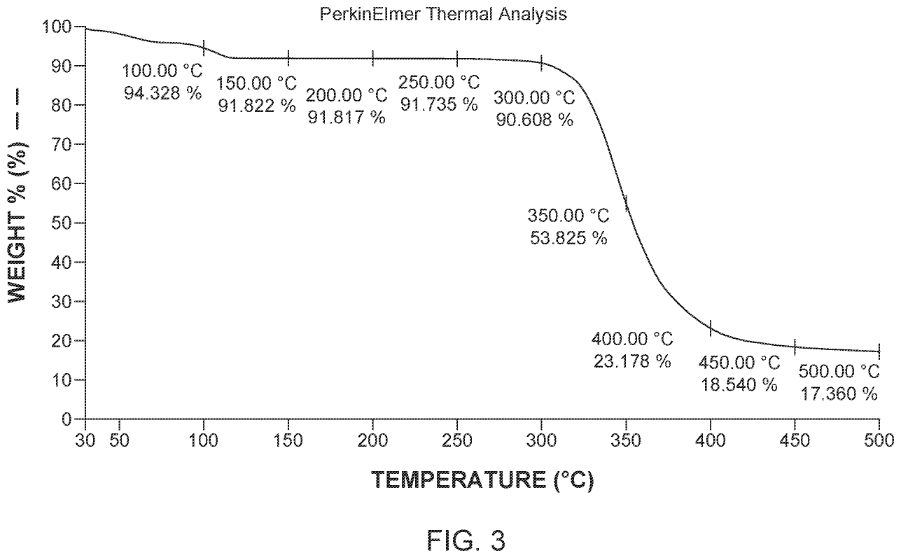

In another embodiment, the present invention provides Ivacaftor polymorphic form RK1 characterized by a Thermogravimetric analysis (TGA) in accordance with FIG. 3.

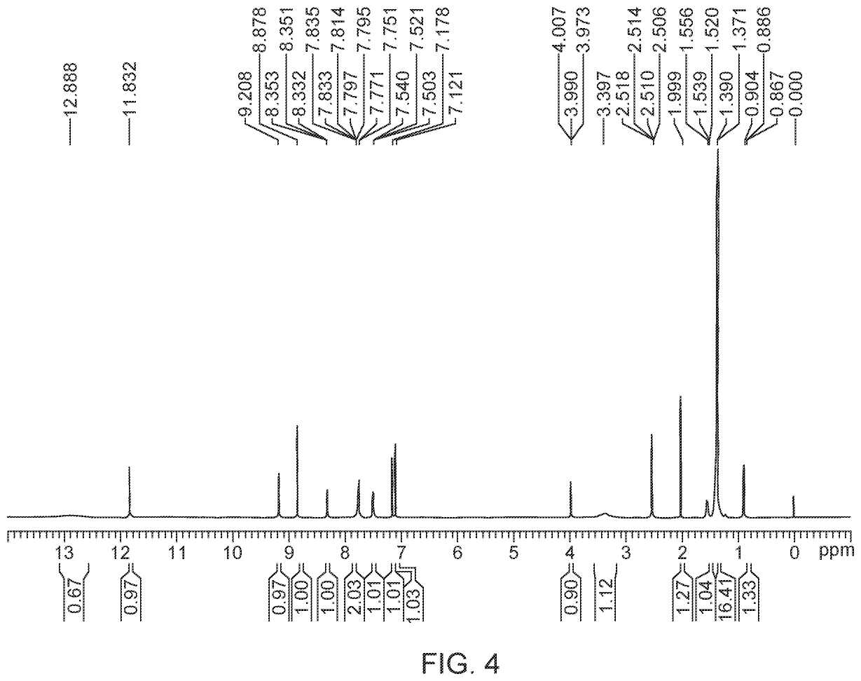

In another embodiment, the present invention provides Ivacaftor polymorphic form RK1 characterized by a Nuclear Magnetic Resonance (NMR) spectrogram in accordance with FIG. 4.

In another embodiment, the present invention provides a process for preparation of Ivacaftor polymorphic form RK1, comprising:
a) dissolving Ivacaftor or a solvate thereof in n-Butyl acetate,
b) heating the reaction mixture,
c) filtering the reaction mixture and cooling the filtrate to −20° C.
d) isolating the solid; and
e) drying the solid at about 85° C. to about 95° C. to obtain Ivacaftor polymorphic form RK1.

In the aforementioned process of Ivacaftor polymorphic form RK1 includes dissolving Ivacaftor or Ivacaftor methanol solvate in n-Butyl acetate at a suitable temperature for example at 50-60° C. The solution was filtered to remove undissolved particulate. Filtrate was further cooled to −20° C. and stirred for 2 hours. Hexane was then added in a slurry and stirred for one hour at 25-30° C., filtered the obtained solid and further dried in a vacuum at 40-50° C. for 12 hours to obtain Ivacaftor polymorphic form RK1.

In another embodiment, the present invention provides Ivacaftor polymorphic form RK2.

In another embodiment, the present invention provides Ivacaftor polymorphic form RK2 characterized by a powder X-Ray diffraction (PXRD) pattern.

In another embodiment, the present invention provides Ivacaftor polymorphic form RK2 characterized by a PXRD pattern having one or more peaks at about 5.08, 6.65, 6.79, 6.93, 7.00, 7.19, 8.19, 8.27, 10.05, 10.69, 12.93, 13.63, 13.72, 13.92, 15.62, 15.75, 17.88, 18.69, 19.64, 20.26, 20.32, 20.73, 21.69, 23.38, 23.67, 25.37, 27.93, 28.11, 28.90±0.2° 2θ.

In another embodiment, the present invention provides a process for preparation of Ivacaftor polymorph form RK2, comprising:
a) dissolving Ivacaftor or Ivacaftor methanol solvate in diethyl ether
b) solution stirred at 25-30° C. for 12 hours.
c) isolating the solid; and
d) drying the solid obtained in step c) at about 35° C. to about 45° C. for 12 hours to obtain Ivacaftor polymorphic form RK2.

In another embodiment, the present invention provides Ivacaftor polymorphic form RK3.

In another embodiment, the present invention provides Ivacaftor polymorphic form RK3 characterized by a powder X-Ray diffraction (PXRD) pattern.

In another embodiment, the present invention provides Ivacaftor polymorphic form RK3 characterized by a PXRD pattern having one or more peaks at about 5.71, 7.18, 7.58, 8.02, 8.28, 8.99, 9.41, 9.78, 10.50, 11.05, 11.83, 11.91, 12.67, 12.88, 13.54, 13.98, 14.76, 15.29, 15.64, 16.19, 16.56, 17.20, 18.08, 18.46, 18.66, 18.86, 19.50, 20.09, 20.62, 22.11, 23.58, 25.54, 26.09±0.2° 2θ.

In another embodiment, the present invention provides a process for preparation of Ivacaftor polymorphic form RK3, comprising:
a) dissolving Ivacaftor or Ivacaftor methanol solvate in anisole
b) stirring the solution at 25-30° C. for 3 hours.
c) isolating the solid; and
d) drying the solid obtained in step c) at about 40° C. to 50° C. for 12 hours to obtain Ivacaftor polymorphic form RK3.

In another embodiment, the present invention provides Ivacaftor polymorphic form RK4. In another embodiment, the present invention provides Ivacaftor polymorphic form RK4 characterized by a powder X-Ray diffraction (PXRD) pattern.

In another embodiment, the present invention provides Ivacaftor polymorphic form RK4 characterized by a PXRD pattern having one or more peaks at about 4.05, 9.57, 9.95, 10.41, 11.76, 12.19, 14.32, 15.17, 18.87, 19.90, 25.33, 26.43±0.2° 2θ.

In another embodiment, the present invention provides a process for preparation of Ivacaftor

Form RK4, comprising:
a) dissolving Ivacaftor or Ivacaftor methanol solvate in 1,2-dimethoxyethane
b) stirring the solution for one hour.
c) adding water to the step b) solution at about 25° C. to about 30° C. and stirring for 48 hours.
d) isolating the solid; and
e) drying the solid obtained in step d) at 40-50° C. to obtain Ivacaftor polymorphic form RK4.

Ivacaftor or Ivacaftor methanol solvate dissolved in 1,2-dimethoxyethane and stirred at room temperature for one hour, then added water to the slurry and stirred for 48 hours. The obtained solid was filtered. Solid further dried in a vacuum oven at 40-50° C. for 12 hours to obtain polymorphic form RK4 of Ivacaftor.

In another embodiment, the present invention provides Ivacaftor polymorphic form RK5.

In another embodiment, the present invention provides Ivacaftor polymorphic form RK5 characterized by a powder X-Ray diffraction (PXRD) pattern.

In another embodiment, the present invention provides Ivacaftor polymorphic form RK5 characterized by a PXRD pattern having one or more peaks at about 5.04, 8.61, 9.52, 9.70, 10.52, 10.61, 12.21, 12.93, 15.50, 16.59, 16.88, 18.54, 19.33, 21.22, 21.89, 22.09, 24.58, 28.27±0.2° 2θ.

In another embodiment. The present invention provides a process for preparation of Ivacaftor polymorphic form RK5, comprising:
a) dissolving Ivacaftor or Ivacaftor methanol solvate in ethoxy ethanol at temperature 25-30° C.
b) stirring for 3 hours at 25-30° C.
c) isolating the solid; and
d) drying the solid obtained in step c) at 40-50° C. to obtain Ivacaftor polymorphic form RK5.

In another embodiment, the present invention provides Ivacaftor polymorphic form RK6.

In another embodiment, the present invention provides Ivacaftor polymorphic form RK6 characterized by a powder X-Ray diffraction (PXRD) pattern.

In another embodiment, the present invention provides Ivacaftor polymorphic form RK6 characterized by a PXRD pattern having one or more peaks at about 6.41, 9.24, 9.42, 10.95, 12.17, 12.70, 14.52, 18.99, 20.74, 21.04, 23.10±0.2° 2θ.

In another embodiment, the present invention provides a process for preparation of Ivacaftor polymorphic form RK6; comprising:
a) dissolving Ivacaftor or Ivacaftor methanol solvate in trifluoroacetic acid at a suitable temperature,
b) adding water to the step a) solution at 25° C. to about 35° C.,
c) isolating the solid; and
d) drying the solid obtained in step c) at 40-50° C. to obtain Ivacaftor polymorphic form RK6.
e) polymorphic form RK6 obtained in step d) dried at 150° C. for 6 hours to obtain polymorphic form RK6H of Ivacaftor.

In the aforementioned process of Ivacaftor polymorphic form RK6 includes dissolving Ivacaftor methanol solvate in Trifluoroacetic acid stirring at room temperature to dissolve it completely. Filtering the solution to remove undissolved particulate. Adding water to the filtered solution at room temperature and stirring for 3 hours to obtain solid. Drying the solid in a vacuum oven at 40-50° C. for 12 hours to obtain polymorphic form RK6 of Ivacaftor. Polymorphic form RK6 of Ivacaftor obtained in above was dried at 150° C. for 6 hours to obtain polymorphic form RK6H of Ivacaftor.

In another embodiment, the present invention provides Ivacaftor polymorphic form RK7.

In another embodiment, the present invention provides Ivacaftor polymorphic form RK7 characterized by a powder X-Ray diffraction (PXRD) pattern.

In another embodiment, the present invention provides Ivacaftor polymorphic form RK7 characterized by a PXRD pattern having one or more peaks at about 4.72, 5.85, 6.04, 6.35, 8.19, 8.62, 9.65, 10.33, 11.03, 11.44, 12.09, 12.73, 13.33, 13.88, 14.34, 15.54, 16.57, 17.72, 18.20, 19.19, 19.64, 20.43, 20.81, 22.03, 23.09, 23.71, 24.91, 26.06, 26.58, 29.42±0.2° 2θ.

In another embodiment, the present invention provides Ivacaftor polymorphic form RK7 characterized by a differential scanning calorimetry (DSC) substantially in accordance with FIG. 11.

In another embodiment, the present invention provides Ivacaftor polymorphic form RK7 characterized by a Thermogravimetric analysis (TGA).

In another embodiment, the present invention provides a process for the preparation of Ivacaftor polymorphic form RK7, comprising:
a) dissolving Ivacaftor or Ivacaftor methanol solvate in Trifluoroacetic acid,
b) adding methyl tertiary butyl ether to the step a) solution at 25° C. to about 35° C.,
c) isolating the solid; and
d) drying the solid obtained in step c) at 40-50° C. to obtain Ivacaftor polymorphic form RK7.

In another embodiment, the present invention provides Ivacaftor polymorphic form RK8.

In another embodiment, the present invention provides Ivacaftor polymorphic form RK8 characterized by a powder X-Ray diffraction (PXRD) pattern.

In another embodiment, the present invention provides Ivacaftor polymorphic form RK8 characterized by a PXRD pattern having one or more peaks at about 4.54, 4.95, 5.35, 6.92, 7.87, 8.07, 8.19, 8.65, 9.11, 9.34, 10.83, 12.44, 14.37, 15.02, 16.01, 17.29, 17.47, 18.25, 18.40, 18.62, 18.84, 19.02, 19.62, 23.44±0.2° 2θ.

In another embodiment, the present invention provides Ivacaftor polymorphic form RK8 characterized by a differential scanning calorimetry (DSC).

In another embodiment, the present invention provides Ivacaftor polymorphic form RK8 characterized by a Thermogravimetric analysis (TGA).

In another embodiment, the present invention provides a process for the preparation of Ivacaftor polymorphic form RK8, comprising:
a) dissolving Ivacaftor or Ivacaftor methanol solvate in chlorobenzene,
b) heating the reaction mixture,
c) cooling the mixture to room temperature and stirring for 2 hours to obtain solid;
d) filtering the obtained solid and drying in vacuum oven at 40-50° C. for 12 hours to obtain Ivacaftor polymorphic form RK8.

In another embodiment, Polymorphic form RK4 of Ivacaftor was dried at 150° C. for 6 hours to obtain polymorphic form RK9H of Ivacaftor.

In another embodiment, the present invention provides Ivacaftor polymorphic form RK9.

In another embodiment, the present invention provides Ivacaftor polymorphic form RK9 characterized by a powder X-Ray diffraction (PXRD) pattern.

In another embodiment, the present invention provides Ivacaftor polymorphic form RK9 characterized by a PXRD pattern having one or more peaks at about 5.31, 5.98, 7.21, 8.02, 8.82, 10.03, 12.04, 12.28, 15.32, 15.74, 17.14, 18.13, 18.60, 18.81, 19.91, 20.20, 20.78, 21.20, 24.65±0.2° 2θ.

In another embodiment, the present invention provides Ivacaftor polymorphic form RK9 characterized by a differential scanning calorimetry (DSC).

In another embodiment, the present invention provides Ivacaftor polymorphic form RK9 characterized by a Thermogravimetric analysis (TGA).

In another embodiment, the present invention provides a process for the preparation of Ivacaftor polymorphic form RK9, comprising;
a) dissolving Ivacaftor or Ivacaftor methanol solvate in diphenyl ether,
b) heating the suspension,
c) isolating the solid; and
d) drying the solid to obtain Ivacaftor polymorphic form RK9.

In another embodiment, the present invention provides Ivacaftor polymorph form RK10.

In another embodiment, the present invention provides Ivacaftor polymorph form RK10 characterized by a powder X-Ray diffraction (PXRD) pattern.

In another embodiment, the present invention provides Ivacaftor polymorph form RK10 characterized by a PXRD pattern having one or more peaks at about 4.99, 5.37, 7.64, 7.96, 8.28, 8.43, 9.34, 10.04, 11.46, 13.93, 14.69, 15.43, 15.70, 16.04, 18.19, 18.92, 23.81±0.2° 2θ.

In another embodiment, the present invention provides a process for the preparation of Ivacaftor form RK10, comprising:
a) dissolving Ivacaftor or Ivacaftor methanol solvate in Bromobenzene,
b) stirring at room temperature for 12 hours,
c) isolating the solid; and
d) drying the solid at 40-50° C. to obtain Ivacaftor polymorphic form RK10.

In another embodiment, the present invention provides Ivacaftor polymorphic form RK11.

In another embodiment, the present invention provides Ivacaftor polymorphic form RK12 characterized by a powder X-Ray diffraction (PXRD) pattern.

In another embodiment, the present invention provides Ivacaftor polymorphic form RK11 characterized by a PXRD pattern having one or more peaks at about 5.86, 6.10, 6.48, 8.44, 9.06, 9.87, 10.42, 11.06, 12.43, 12.90, 13.42, 14.11, 14.85, 14.95, 16.49, 16.71, 16.97, 17.22, 17.93, 18.07, 18.80, 18.99, 19.56, 19.81, 20.29, 20.68, 21.01, 21.90, 22.14, 22.50, 22.93, 23.26, 24.13, 24.57, 25.77, 26.05, 26.39, 28.39, 30.18, 30.83±0.2° 2θ.

In another embodiment, the present invention provides a process for the preparation of Ivacaftor polymorphic form RK11, comprising:
a) dissolving Ivacaftor or Ivacaftor methanol solvate in Iodobenzene,
b) stirring the reaction mixture at room temperature for 12 hours.
c) isolating the solid; and
d) drying the solid at 40° C. to 50° C. to obtain Ivacaftor polymorphic form RK11.

In another embodiment, the present invention provides Ivacaftor polymorphic form RK12.

In another embodiment, the present invention provides Ivacaftor polymorphic form RK12 characterized by a powder X-Ray diffraction (PXRD) pattern.

In another embodiment, the present invention provides Ivacaftor polymorphic form RK12 characterized by a PXRD pattern having one or more peaks at about 4.61, 9.12, 9.64, 13.33, 14.00, 19.26, 21.82, 23.35±0.2° 2θ.

In another embodiment, the present invention provides a process for the preparation of Ivacaftor polymorphic form RK12, comprising:
a) dissolving Ivacaftor or Ivacaftor methanol solvate in Benzonitrile,
b) stirring at room temperature 2 hours,
c) adding hexane into slurry at room temperature and stirring for one hour.
c) filtering and drying the obtained solid.
d) further drying the solid at 40° C. to 50° C. to obtain Ivacaftor polymorphic form RK12.

In another embodiment, the present invention provides Ivacaftor polymorphic form RK13.

In another embodiment, the present invention provides Ivacaftor polymorphic form RK13 characterized by a powder X-Ray diffraction (PXRD) pattern.

In another embodiment, the present invention provides Ivacaftor polymorphic form RK13 characterized by a PXRD pattern having one or more peaks at about 5.25, 7.17, 8.26, 9.12, 10.08, 10.66, 11.14, 12.93, 13.06, 13.72, 13.91, 15.72, 18.74, 19.60, 20.29, 20.80, 23.37, 28.06±0.2° 2θ.

In another embodiment, the present invention provides a process for the preparation of Ivacaftor polymorphic form RK13, comprising:
a) dissolving Ivacaftor or Ivacaftor methanol solvate in 1,1,2-trichloroethane,
b) stirring the reaction mixture for 12 hours at room temperature,
c) isolating the solid; and
d) drying the solid at 40° C. to 50° C. to obtain Ivacaftor polymorphic form RK13.

In another embodiment, the present invention provides Ivacaftor polymorphic form RK14.

In another embodiment, the present invention provides Ivacaftor polymorphic form RK14 characterized by a powder X-Ray diffraction (PXRD) pattern.

In another embodiment, the present invention provides Ivacaftor polymorphic form RK14 characterized by a PXRD pattern having one or more peaks at about 4.46, 6.10, 6.66, 7.56, 10.76, 12.19, 17.20, 18.37±0.2° 2θ.

In another embodiment, the present invention provides a process for the preparation of Ivacaftor polymorphic form RK14, comprising:
a) dissolving Ivacaftor or Ivacaftor methanol solvate in Carbon tetrachloride,
b) heating the reaction mixture to 75° C.,
c) filtering the solution at 40° C. to remove undissolved particulate.
d) cooling the filtered reaction mixture to 30° C. and stirring for 2 hours.
d) drying the solid under vacuum oven at 40° C. to 50° C. to obtain Ivacaftor polymorphic form RK14.

In another embodiment, the present invention provides Ivacaftor polymorphic form RK15.

In another embodiment, the present invention provides Ivacaftor polymorphic form RK15 characterized by a powder X-Ray diffraction (PXRD) pattern.

In another embodiment, the present invention provides Ivacaftor polymorphic form RK15 characterized by a PXRD pattern having one or more peaks at about 7.74, 8.18, 8.66, 9.39, 9.60, 11.40, 11.64, 13.80, 14.43, 15.15, 15.45, 15.53, 16.12, 16.56, 17.11, 17.62, 17.86, 18.10, 19.39, 19.91, 20.25, 20.57, 21.05, 21.21, 22.04, 22.13, 23.53, 23.62, 23.74, 24.81±0.2° 2θ.

In another embodiment, the present invention provides a process for the preparation of Ivacaftor polymorphic form RK15, comprising:
a) dissolving Ivacaftor or Ivacaftor methanol solvate in 2,2-dimethoxypropane,
b) stirring the reaction mixture for 12 hours at room temperature,
c) isolating the solid; and d) drying the solid at 40° C. to 50° C. for 12 hours to obtain Ivacaftor polymorphic form RK15.

In another embodiment, the present invention provides Ivacaftor polymorphic form RK16. In another embodiment, the present invention provides Ivacaftor polymorphic form RK16 characterized by a powder X-Ray diffraction (PXRD) pattern.

In another embodiment, the present invention provides Ivacaftor polymorphic form RK16 characterized by a PXRD pattern having one or more peaks at about 5.06, 15.47, 16.39, 16.84, 17.97, 20.56, 22.36±0.2° 2θ.

In another embodiment, the present invention provides a process for the preparation of Ivacaftor polymorphic form RK16, comprising:
a) dissolving Ivacaftor or Ivacaftor methanol solvate in Methyl Isopropyl Ketone,
b) stirring the reaction mixture for 3 hours at room temperature,
c) isolating the solid; and
d) drying the solid at 40° C. to 50° C. for 12 hours to obtain Ivacaftor polymorphic form RK16.

In another embodiment, the present invention provides Ivacaftor polymorphic form RK17.

In another embodiment, the present invention provides Ivacaftor polymorphic form RK17 characterized by a powder X-Ray diffraction (PXRD) pattern.

In another embodiment, the present invention provides Ivacaftor polymorphic form RK17 characterized by a PXRD pattern having one or more peaks at about 5.35, 7.99, 9.07, 11.17, 11.52, 12.23, 12.64, 13.42, 15.08, 15.93, 16.42, 17.53, 19.14, 19.79, 21.52, 43.54±0.2° 2θ.

In another embodiment, the present invention provides a process for the preparation of Ivacaftor polymorphic form RK17, comprising:
a) dissolving Ivacaftor or Ivacaftor methanol solvate in 2-methyl THF,
b) stirring the reaction mixture for 3 hours at room temperature,
c) isolating the solid; and
d) drying the solid at 40° C. to 50° C. for 12 hours to obtain Ivacaftor polymorphic form RK17.

### EXAMPLES

The following non limiting examples illustrate specific embodiments of the present invention. They are not intended to limit the scope of the present invention in any way.

### Example 1: Preparation of Ivacaftor Polymorphic Form RK1

n-Butyl acetate (7 mL) was charged into a round bottom flask. Ivacaftor methanol solvate (1 g) was then introduced into a round bottom flask and stirred. Reaction mixture was heated at 60° C. temperature to dissolve it completely. The solution was filtered at 40° C. to remove undissolved particulate. Filtrate was further cooled to −20° C. and stirred for 2 hrs. Hexane (20 mL) was then added into slurry at room temperature and stirred for 1 hr to obtain form RK1. Obtained solid was filtered and suck dried well. Solid was further dried in vacuum oven at 40-50° C. for 12 hours to obtain polymorphic form RK1 of Ivacaftor.

### Example 2: Preparation of Ivacaftor Polymorphic Form RK2

Diethyl ether (10 mL) was charged into a round bottom flask. Ivacaftor methanol solvate (1 g) was then introduced into a round bottom flask and stirred at room temperature for 12 hrs. Obtained solid was filtered and suck dried well. Solid was further dried in vacuum oven at 35-45° C. for 12 hours to obtain polymorphic form RK2 of Ivacaftor.

### Yield: 0.79 g

### Example 3: Preparation of Ivacaftor Polymorphic Form RK3

Anisole (20 mL) was charged into a round bottom flask. Ivacaftor methanol solvate (1 g) was then introduced into a round bottom flask and stirred at room temperature for 3 hrs. Obtained solid was filtered and suck dried well. Solid was further dried in vacuum oven at 40-50° C. for 12 hours to obtain polymorphic form RK3 of Ivacaftor.

### Yield: 0.72 g

### Example 4: Preparation of Ivacaftor Polymorphic Form RK4

1,2-dimethoxyethane (20 mL) was charged into a round bottom flask. Ivacaftor methanol solvate (1 g) was then introduced into a round bottom flask and stirred at room temperature for 1 hour. Water (20 mL) was then added into slurry at room temperature and stirred for 48 hrs. Obtained solid was filtered and suck dried well. Solid was further dried in vacuum oven at 40-50° C. for 12 hours to obtain polymorphic form RK4 of Ivacaftor.

### Yield: 0.93 g

### Example 5: Preparation of Ivacaftor Polymorphic Form RK5

2-Ethoxy ethanol (10 mL) was charged into a round bottom flask. Ivacaftor methanol solvate (1 g) was then introduced into a round bottom flask and stirred at room temperature for 3 hrs. Obtained solid was filtered and suck dried well. Solid was further dried in vacuum oven at 40-50° C. for 12 hours to obtain polymorphic form RK5 of Ivacaftor.

### Yield: 0.62 g

### Example 6: Preparation of Ivacaftor Polymorphic Form RK6

Trifluoroacetic acid (TFA) (10 mL) was charged into a round bottom flask. Ivacaftor methanol solvate (1 g) was then introduced into a round bottom flask and stirred at room temperature to dissolve it completely. The solution was filtered to remove undissolved particulate. Water (40 mL) was then added in filtered solution at room temperature and stirred for 3 hrs. Obtained solid was filtered and suck dried well. Solid was further dried in vacuum oven at 40-50° C. for 12 hours to obtain polymorphic form RK6 of Ivacaftor.

### Yield: 0.87 g

Polymorphic form RK6 of Ivacaftor obtained in the Example was dried at 150° C. for 6 hours to obtain polymorphic form RK6H of Ivacaftor.

### Example 7: Preparation of Ivacaftor Polymorphic Form RK7

Trifluoroacetic acid (TFA) (7 mL) was charged into a round bottom flask. Ivacaftor methanol solvate (1 g) was then introduced into a round bottom flask and stirred at room temperature to dissolve it completely. The solution was filtered to remove undissolved particulate. Methyl tertiary butyl ether (MTBE) (15 mL) was then added in filtered solution at room temperature and stirred for 1 hr. Obtained solid was filtered and suck dried well. Solid was further dried in vacuum oven at 40-50° C. for 12 hours to obtain polymorphic form RK7 of Ivacaftor.

### Yield: 0.69 g

### Example 8: Preparation of Ivacaftor Polymorphic Form RK8

Chlorobenzene (7 mL) was charged into a round bottom flask. Ivacaftor methanol solvate (1 g) was then introduced into a round bottom flask and stirred. Reaction mixture was heated at 80° C. temperature to dissolve it completely. The solution was filtered at 50° C. to remove undissolved particulate. Filtrate was further cooled to RT and stirred for 2 hrs. Obtained solid was filtered and suck dried well. Solid was further dried in vacuum oven at 40-50° C. for 12 hours to obtain polymorphic form RK8 of Ivacaftor.

Yield: 0.89 g. Polymorphic form RK4 of Ivacaftor obtained in the Example 4 was dried at 150° C. for 6 hours to obtain polymorphic form RK9H of Ivacaftor.

### Example 9: Preparation of Ivacaftor Polymorphic Form RK9

Diphenyl ether (20 mL) was charged into a round bottom flask. Ivacaftor methanol solvate (1 g) was then introduced into a round bottom flask and stirred at room temperature for 12 hrs. Obtained solid was filtered and suck dried well. Solid was further dried in vacuum oven at 40-50° C. for 12 hours to obtain polymorphic form RK9 of Ivacaftor.

### Yield: 0.9 g

### Example 10: Preparation of Ivacaftor Polymorphic Form RK10

Bromobenzene (20 mL) was charged into a round bottom flask. Ivacaftor methanol solvate (1 g) was then introduced into a round bottom flask and stirred at room temperature for 12 hrs. Obtained solid was filtered and suck dried well. Solid was further dried in vacuum oven at 40-50° C. for 12 hours to obtain polymorphic form RK10 of Ivacaftor. PXRD analysis shows that the solid obtained is consistent with polymorph Form RK10.

### Yield: 0.86 g

### Example 11: Preparation of Ivacaftor Polymorphic Form RK11

Iodobenzene (10 mL) was charged into a round bottom flask. Ivacaftor methanol solvate (1 g) was then introduced into a round bottom flask and stirred at room temperature for 12 hrs. Obtained solid was filtered and suck dried well. Solid was further dried in vacuum oven at 40-50° C. for 12 hours to obtain polymorphic form RK11 of Ivacaftor.

### Yield: 0.88 g

### Example 12: Preparation of Ivacaftor Polymorphic Form RK12

Benzonitrile (20 mL) was charged into a round bottom flask. Ivacaftor methanol solvate (1 g) was then introduced into a round bottom flask and stirred at room temperature for 2 hrs. Hexane (20 mL) was then added into the slurry at room temperature and stirred for 1 hr. The obtained solid was filtered and suck dried well. Solid was further dried in vacuum oven at 40-50° C. for 12 hours to obtain polymorphic form RK12 of Ivacaftor.

### Yield: 0.91 g

### Example 13: Preparation of Ivacaftor Polymorphic Form RK13

1,1,2-trichloroethane (10 mL) was charged into a round bottom flask. Ivacaftor methanol solvate (1 g) was then introduced into a round bottom flask and stirred at room temperature for 12 hrs. Obtained solid was filtered and suck dried well. Solid was further dried in vacuum oven at 40-50° C. for 12 hours to obtain polymorphic form RK13 of Ivacaftor.

### Yield: 0.93 g

### Example 14: Preparation of Ivacaftor Polymorphic Form RK14

Carbon tetrachloride (10 mL) was charged into a round bottom flask. Ivacaftor methanol solvate (1 g) was then introduced into a round bottom flask and stirred. Reaction mixture was heated at 75° C. temperature to dissolve it completely. The solution was filtered at 40° C. to remove undissolved particulate. Filtrate was further cooled to 30° C. and stirred for 2 hrs. Obtained solid was filtered and suck dried well. Solid was further dried in vacuum oven at 40-50° C. for 12 hours to obtain polymorphic form RK14 of Ivacaftor.

### Yield: 0.83 g

### Example 15: Preparation of Ivacaftor Polymorphic Form RK15

2,2-dimethoxypropane (20 mL) was charged into a round bottom flask. Ivacaftor methanol solvate (1 g) was then introduced into a round bottom flask and stirred at room temperature for 12 hrs. Obtained solid was filtered and suck dried well. Solid was further dried in vacuum oven at 40-50° C. for 12 hours to obtain polymorphic form RK15 of Ivacaftor.

### Yield: 0.91 g

### Example 16: Preparation of Ivacaftor Polymorphic Form RK16

Methyl isopropyl ketone (20 mL) was charged into a round bottom flask. Ivacaftor methanol solvate (1 g) was then introduced into a round bottom flask and stirred at room temperature for 3 hrs. Obtained solid was filtered and suck dried well. Solid was further dried in vacuum oven at 40-50° C. for 12 hours to obtain polymorphic form RK16 of Ivacaftor.

### Yield: 0.88 g

### Example 17: Preparation of Ivacaftor Polymorphic Form RK17

2-Methyl THF (10 mL) was charged into a round bottom flask. Ivacaftor methanol solvate (1 g) was then introduced into a round bottom flask and stirred at room temperature for 3 hrs. Obtained solid was filtered and suck dried well. Solid was further dried in vacuum oven at 40-50° C. for 12 hours to obtain polymorphic form RK17 of Ivacaftor.

### Yield: 0.76 g

### Example 18: Preparation of Ivacaftor Polymorphic Form RK1

4-Oxo-1,4-dihydroquinoline-3-carboxylic acid (10 g, 1.0 eq.), Dimethylformamide (30 mL, 3 vol.) and 5-Amino-2,4-di-tertiary-butylphenol hydrochloride (11.73, 0.860 eq.) were charged into RBF at 25-35° C. Triethylamine (22.5 mL, 2.25 vol.) was charged followed by anhydrous 1-Hydroxybenzotriazole (11 g, 1.54 eq.) and 1-Ethyl-3-(3′-dimethylaminopropyl)carbodiimide hydrochloride (19.26 g, 1.9 eq.) into reaction mass at 25-35° C. Reaction mass temperature was raised to 45-50° C. and stirred for 4-5 hours at 45-50° C. The progress of the reaction was monitored by TLC. After completion of reaction, reaction mass was quenched with water (50 mL, 5 vol.) at 25-35° C. and diluted with Ethyl acetate (50 mL, 5 vol.). Layers were separated and aqueous layer was extracted with Ethyl acetate (30 mL, 3 vol.). Organic layers were combined and treated with charcoal. Solvent was distilled and co-distilled with Methanol (10 mL, 1 vol.) followed by n-Butyl acetate (1 vol.). To the resultant mass, n-Butyl acetate (200 mL, 20 vol.) was charged and heated to 70° C. The reaction mass was cooled to 25-35° C. and seeding material (RK1, 0.2 g) was added. Further the reaction mass was cooled to −20° C. and stirred for 4-5 hours and filtered. The wet material was washed with n-Butyl acetate (20 mL, 2 vol.) and dried to obtain polymorphic form RK1 of Ivacaftor.

### Yield: 7.1 g

### Example 19: Preparation of Ivacaftor Polymorphic Form RK1

4-Oxo-1,4-dihydroquinoline-3-carboxylic acid (10 g, 1.0 eq.), Dimethylformamide (30 mL, 3 vol.) and 5-Amino-2,4-di-tertiary-butylphenol hydrochloride (11.73, 0.860 eq.) were charged into RBF at 25-35° C. Triethylamine (22.5 mL, 2.25 vol.) was charged followed by anhydrous 1-Hydroxybenzotriazole (11 g, 1.54 eq.) and 1-Ethyl-3-(3′-dimethylaminopropyl)carbodiimide hydrochloride (19.26 g, 1.9 eq.) into reaction mass at 25-35° C. Reaction mass temperature was raised to 45-50° C. and stirred for 4-5 hours at 45-50° C. The progress of the reaction was monitored by TLC. After completion of reaction, reaction mass was quenched with water (50 mL, 5 vol.) at 25-35° C. and diluted with Ethyl acetate (50 mL, 5 vol.). Layers were separated and aqueous layer was extracted with Ethyl acetate (30 mL, 3 vol.). Organic layers were combined and treated with charcoal. Solvent was distilled and co-distilled with Methanol (10 mL, 1 vol.) followed by n-Butyl acetate (1 vol.). To the resultant mass, n-Butyl acetate (150 mL, 15 vol.) was charged and heated to 70° C. The reaction mass was cooled to 25-35° C. and seeding material (RK1, 0.2 g) was added. Further the reaction mass was cooled to −20° C. and Hexane (450 mL) was added. The resultant mixture was stirred for 24-25 hours and filtered. The wet material was washed with n-Butyl acetate (20 mL, 2 vol.) and dried to obtain polymorphic form RK1 of Ivacaftor.

### Yield: 7.0 g

### Example 20: Preparation of Ivacaftor Methanol Solvate

4-Oxo-1,4-dihydroquinoline-3-carboxylic acid (10 g, 1.0 eq.), Dimethylformamide (30 mL, 3 vol.) and 5-Amino-2,4-di-tertiary-butylphenol hydrochloride (11.73, 0.860 eq.) were charged into RBF at 25-35° C. Triethylamine (22.5 mL, 2.25 vol.) was charged followed by anhydrous 1-Hydroxybenzotriazole (11 g, 1.54 eq.) and 1-Ethyl-3-(3′-dimethylaminopropyl)carbodiimide hydrochloride (19.26 g, 1.9 eq.) into reaction mass at 25-35° C. Reaction mass temperature was raised to 45-50° C. and stirred for 4-5 hours at 45-50° C. The progress of the reaction was monitored by TLC. After completion of reaction, reaction mass was quenched with water (50 mL, 5 vol.) at 25-35° C. and diluted with Ethyl acetate (50 mL, 5 vol.). Layers were separated and aqueous layer was extracted with Ethyl acetate (30 mL, 3 vol.). Organic layers were combined and treated with charcoal. Solvent was distilled and co-distilled with Methanol (10 mL, 1 vol.). Methanol (50 mL) charged into reaction mass. Reaction mass stirred for 2-3 hours at 25-35° C. Reaction mass was filtered and washed with Methanol (5 mL). The obtained solid was dried to get Ivacaftor methanol solvate.

### Yield: 10.0 g

The present invention will be further illustrated with reference to the following examples which aid in understanding, but which are not to be construed as limitations thereof.

Without wishing to be bound to a theory, the process described in the present invention is believed to be an improved process for the preparation of Ivacaftor new polymorphs, which is commercially scalable, economical, stable and provides Ivacaftor with improved yield along with high purity.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

We claim:

1. A crystalline n-butyl acetate solvate form of Ivacaftor of formula (I)

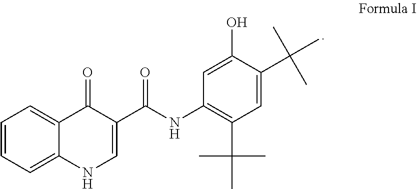 [SMILES: CC(C)(C)c1cc(C(C)(C)C)c(NC(=O)c2c[nH]c3ccccc3c2=O)cc1O]

2. The crystalline n-butyl acetate solvate as claimed in claim 1, referred to as Form RK1 is characterized by a PXRD pattern having one or more peaks at about 4.8, 5.1, 8.3, 9.6, 11.0, 13.2, 19.5, 22.6±0.2° 2θ.

3. The crystalline n-butyl acetate solvate as claimed in claim 2, wherein the crystalline Form RK1 is further characterized by X-ray powder diffraction pattern having one or more peaks at about 4.8, 5.1, 7.8, 8.3, 9.6, 11.0, 13.2, 13.8, 18.7, 19.5, 19.7, 20.8, 22.6±0.2° 2θ.

4. A process for the preparation of crystalline n-butyl acetate solvate of Ivacaftor RK-1 comprise,
a) adding Ivacaftor or a solvate thereof in n-Butyl acetate,
b) heating the reaction mixture,
c) filtering the reaction mixture and cooling the filtrate to −20° C.
d) optionally adding anti-solvent and isolating the solid.
e) drying the solid to obtain Ivacaftor polymorphic form RK1.

5. The process as claimed in claim 4, wherein solvate used in step a) is Ivacaftor methanol solvate.

6. The process as claimed in claim 4, wherein in step (b) heating is carried out at a temperature of 50 to 70° C. for a period of 2 hours.

7. An improved process for the preparation of Ivacaftor formula I or a pharmaceutically acceptable salt thereof, comprising the steps of,
a) coupling of compound of formula ICA-03 with compound of formula ICB-02 using suitable reagent and base in presence of solvent to form Ivacaftor formula I

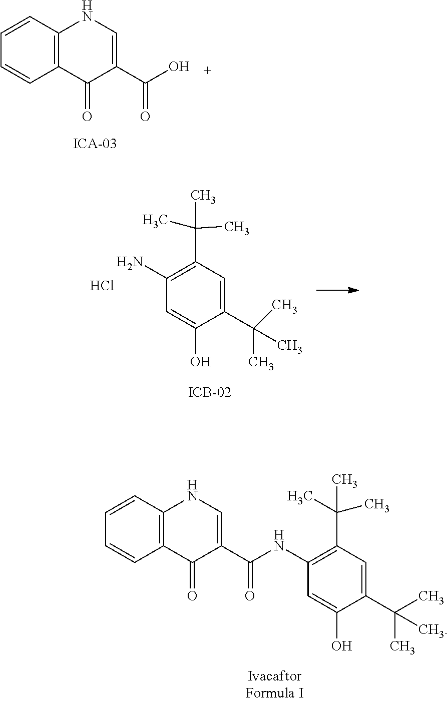 [SMILES: O=C(O)c1c[nH]c2ccccc2c1=O ; Cl ; CC(C)(C)c1cc(C(C)(C)C)c(O)cc1N ; CC(C)(C)c1cc(C(C)(C)C)c(NC(=O)c2c[nH]c3ccccc3c2=O)cc1O]

8. The process as claimed in claim 7, wherein the suitable reagent used in step-a) is selected from group consisting of 1-[bis(dimethylamino)methylene]-1H-1,2,3-triazolo[4,5-b]pyridinium-3-oxide hexafluorophosphate (HATU), N,N,N′,N′-tetramethyl-O-(1H-benzotriazol-1-yl)uranium hexafluorophosphate (HBTU), Hydroxybenzotriazole (HOBt), N,N′-Dicyclohexylcarbodimide (DCC), 1-(3-dimethylaminopropyl)-3-ethylcarbodiimide hydrochloride (EDC.HCl), Propanephosphonic acid anhydride (PPAA, T3P), O-(1H-6-chlorobenzotriazole-1-yl)-1,1,3,3-tetramethyluronium hexafluorophosphate (HCTU) or the mixture thereof.

9. The process as claimed in claim 7, wherein the suitable solvent used in step a) is selected from group consisting of water, sulfoxides, alcohols, halogenated hydrocarbons, ethers, esters, amides, hydrocarbons such as dimethyl sulfoxide (DMSO), methanol, ethanol, n-propanol, isopropanol, n-butanol, dichloromethane (DCM), chloroform, dichloroethane, chlorobenzene, diethyl ether, methyl tert-butyl ether (MTBE), diisopropyl ether, tetrahydrofuran (THF), 2-methyltetrahydrofuran, dioxane, methyl acetate, ethyl acetate, butyl acetate, isopropyl acetate, N,N-dimethylformamide (DMF), N,N-dimethylacetamide (DMAc), hexane, heptane, octane, cyclohexane, cyclopentane, toluene, xylene or the mixture thereof.

10. The process as claimed in claim 7, wherein the suitable base used during condensation in step a) is selected from inorganic or organic base, wherein inorganic base is selected from alkali or alkaline earth metal hydrides, hydroxides, carbonates, bicarbonates, sodium with liquid ammonia, sodamide or like and mixture thereof; organic bases may be selected from diisopropylethylamine, triethylamine, tributylamine, or the mixture thereof, preferably, base used is triethylamine.
